(19) United States
(12) Patent Application Publication
(10) Pub. No.: US 2004/0010235 A1
(43) Pub. Date: Jan. 15, 2004

(54) Anti-reflux syringe

(76) Inventors: Eugene E. Weilbacher, Chesterfield, MO (US); Bradley M. Stewart, O'Fallon, MO (US)

Correspondence Address: BROWN, RUDNICK, BERLACK & ISRAELS, LLP., BOX IP, 18TH FLOOR, ONE FINANCIAL CENTER, BOSTON, MA, 02111, US

(21) Appl. No.: 10/194,842

(22) Filed: Jul. 11, 2002

(51) Int. Cl. A61M 5/315

(52) U.S. Cl. 604/218

(57) ABSTRACT

A syringe is provided that includes a barrel including a nozzle having an interior opening. A plunger rod of the syringe has a first end and a second end. The second end includes an extension dimensioned to ensure that a member, disposed with the second end and enclosing the extension, abuts the interior opening. The barrel may include a distal wall that supports the nozzle and the plunger rod may include a correspondingly configured distal face. The extension may include a longitudinal length such that upon engagement of the member with the interior opening, the length of the extension prevents compressive deformation of the member between the distal wall and the distal face. The member can have a transverse wall and a cylindrical sidewall extending along the second end of the plunger rod. The transverse wall may have a substantially uniform thickness. The extension defines a distal portion which may be dimensioned for selective engagement of the member with the interior opening.

### BACKGROUND

1. Technical Field

The present disclosure generally relates to the field of medical assemblies for the administration of fluids, and more particularly, to a syringe that prevents backflow of fluids.

2. Description of the Related Art

Medical needle assemblies, such as, for example, syringes are well known for flushing intravenous catheters, administering fluid injections, etc. Existing syringe products include permanent needle syringes, attachable needle hub syringes, luer fitting syringes, etc.

Flushing of intravenous catheters is required because the catheters are in communication with blood vessels and as such are subject to obstruction from blood clotting, particulate matter obstruction, etc. To maintain patency in a catheter line and prevent obstruction, periodic flushing of the line is performed. Standard syringes are typically employed for flushing the lines. These syringes may use needles for this procedure or may alternatively be needleless whereby a luer connector facilitates attachment to a catheter.

During a procedure, the typical design of standard syringes is such that after the entire content of fluid in the syringe is expelled, a small percentage of that fluid may be drawn back into the syringe. If solution is drawn back into the syringe, then blood is consequently drawn or refluxes back into the catheter. Blood in the catheter increases the chances of clotting.

The drawback of reflux associated with the standard syringe can be due to the interaction of the syringe components. Tapered areas of a plunger tip and a barrel of the syringe have corresponding geometries that do not perfectly match. Thus, void areas are created at the interface of these syringe components. In use, a resilient rubber plunger tip deforms to the shape of the barrel. The rubber material of the plunger tip fills the void areas as fluid is expelled from the syringe to effectively seal the barrel. After expulsion, the plunger tip rebounds to its original shape. This rebound action draws fluid back through the syringe filling void areas and drawing blood back into the catheter.

To avoid the associated drawbacks of syringe reflux, medical personnel administering flushing of a catheter line must employ a positive pressure technique for maintaining positive pressure within a fluid line of the catheter. The positive pressure technique includes keeping a forward motion on the syringe plunger as the needle, cannula or luer connector is removed from a catheter access site. Positive pressure in the catheter line prevents backflow of fluids. Success of this technique is dependent on the skill level of the medical personnel and creates additional tasks during a procedure. Thus, this technique is not a preferred solution to reflux.

Attempts have also been made to overcome reflux by employing valve systems in the catheter line. However, these type of devices may not reliably seal the line to prevent leakage and can disadvantageously result in high production costs due to their complexity.

Therefore, it would be desirable to overcome the disadvantages and drawbacks of the prior art with a syringe that includes a plunger rod having an extension that significantly reduces reflux of fluids. It would be highly desirable if the extension cooperates with a nozzle of the syringe to significantly reduce rebound of the plunger rod. It is contemplated that the syringe is easily and efficiently manufactured and assembled.

### SUMMARY

Accordingly, a syringe is provided that includes a plunger rod having an extension that significantly reduces reflux of fluids to overcome the disadvantages and drawbacks of the prior art. Desirably, the extension cooperates with a nozzle of the syringe to significantly reduce rebound of the plunger rod. The syringe is easily and efficiently manufactured and assembled. The present disclosure resolves related disadvantages and drawbacks experienced in the art.

The syringe greatly reduces rebound of a plunger as it interacts with the barrel as fluid is expelled. This is accomplished by adding a cylindrical extension to the distal end of the plunger rod. This cylindrical extension is of an appropriate length and diameter to assure the center of the plunger tip always engages the circular area around the opening where fluid flows from the syringe barrel. This advantageously improves reliability during medical procedures.

In one particular embodiment, a syringe is provided in accordance with the principles of the present disclosure. The syringe includes a barrel having a nozzle with an interior opening. A plunger rod has a first end and a second end. The second end includes an extension dimensioned to ensure that a member, disposed with the second end and the extension, abuts the interior opening. The interior opening may have a cylindrical configuration. The extension can be dimensioned for receipt by the interior opening in an abutting relationship. The extension may be monolithically formed with the second end of the plunger rod, or alternatively, the extension may be integrally connected with the second end of the plunger rod. The barrel may include a distal wall that supports the nozzle and the plunger rod can include a correspondingly configured distal face.

The extension may include a longitudinal length and diameter such that upon engagement of the member with the interior opening, the length and diameter of the extension prevents compressive deformation of the member between the distal wall and the distal face. The second end of the plunger rod may have a tapered configuration for supporting the extension. The member can have a transverse wall and a cylindrical sidewall extending along the second end of the plunger rod. The transverse wall may be tapered. The transverse wall may have a substantially uniform thickness. Desirably, the member is at least partially flexible.

In an alternate embodiment, the syringe includes a barrel extending from a proximal end to a distal end thereof. The distal end includes a wall portion tapered to support a nozzle. The nozzle defines an interior opening. A plunger rod is configured for slidable movement within the barrel and has a first end and a second end. The second end includes an extension projecting distally therefrom. The extension defines a distal portion and a member defining a transverse wall and a side wall extending along the second end of the plunger rod. The transverse wall encloses the extension. The distal portion of the extension is dimensioned to provide selective engagement of the member with the interior opening. The second end of the plunger rod may have a tapered distal face.

In another alternate embodiment, the syringe includes a barrel including a nozzle having an interior opening. A member is disposed with a distal end of a plunger rod being movable relative to the barrel. The member includes an inner extension configured to engage the distal end and dimensioned to ensure that the member abuts the interior opening. The inner extension may include a longitudinal length and diameter such that upon engagement of the member with the interior opening, the length and diameter of the extension prevents compressive deformation of the member between a distal wall of the barrel and a distal face of the plunger rod.

### BRIEF DESCRIPTION OF THE DRAWINGS

The objects and features of the present disclosure, which are believed to be novel, are set forth with particularity in the appended claims. The present disclosure, both as to its organization and manner of operation, together with further objectives and advantages, may be best understood by reference to the following description, taken in connection with the accompanying drawings wherein:

FIG. 1 is a cross-sectional view in part elevation, of a syringe, in accordance with the principles of the present disclosure;

FIG. 2 is a cross-sectional view in part elevation, of the syringe shown in FIG. 1 with parts separated;

FIG. 3 is an enlarged side view in part elevation, of the indicated area of detail shown in FIG. 1;

FIG. 4 is an enlarged side view of an alternate embodiment of the distal end of the plunger rod shown in FIG. 1;

FIG. 5 is an enlarged bottom view of an alternate embodiment of a plunger tip member in accordance with the principles of the present disclosure; and

FIG. 6 is a side cross-sectional view of the plunger tip member shown in FIG. 5.

### DETAILED DESCRIPTION OF EXEMPLARY EMBODIMENTS

The exemplary embodiments of the syringe and methods of operation disclosed are discussed in terms of medical assemblies for the administration of fluids, and more particularly, in terms of a syringe that significantly reduces backflow or reflux of fluids including blood, body fluids, flush solution, medication, etc. It is contemplated that the syringe of the present disclosure is employed for flushing intravenous catheters. It is envisioned that the present disclosure also finds application for the injection of preventive medications, medicaments, etc., as well as injections employed during procedures relating to phlebotomy, dental, orthopedic, digestive, intestinal, urinary, veterinary types, etc., to a subject. It is further envisioned that the principles relating to the syringe disclosed may be employed with for example, permanent needle, attachable needle, needleless and luer fitting syringes.

In the discussion which follows, the term “proximal” will refer to the portion of a structure which is closer to the practitioner, while the term “distal” will refer to the portion which is further from the practitioner. As used herein, the term “subject” refers to a patient undergoing a procedure that employs a syringe. According to the present disclosure, the term “practitioner” refers to an individual flushing an intravenous catheter, administering an injection, etc., or otherwise employing a syringe, and may include support personnel.

The component parts of the syringe are fabricated from materials suitable for catheter flushing, medication injections, etc., such as, for example, polymerics or metals, such as stainless steel, depending on the particular syringe application and/or preference of a practitioner. Semi-rigid and rigid polymerics are contemplated for fabrication, as well as resilient materials, such as molded medical grade polypropylene. One skilled in the art, however, will realize that other materials and fabrication methods suitable for assembly and manufacture, in accordance with the present disclosure, also would be appropriate.

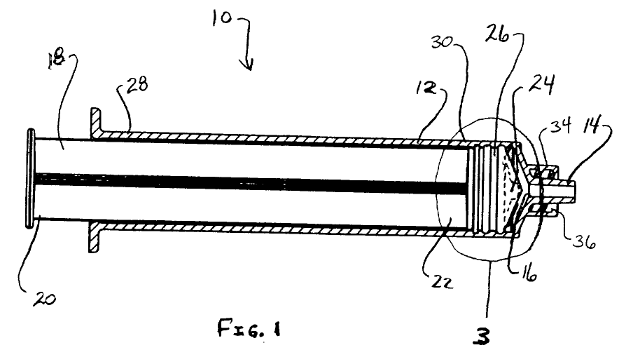

Reference will be now be made in detail to the exemplary embodiments of the disclosure, which are illustrated in the accompanying figures. Turning now to the figures wherein like components are designated by like reference numerals throughout the several views and initially to FIGS. 1-3, there is illustrated a syringe 10, in accordance with the principles of the present disclosure.

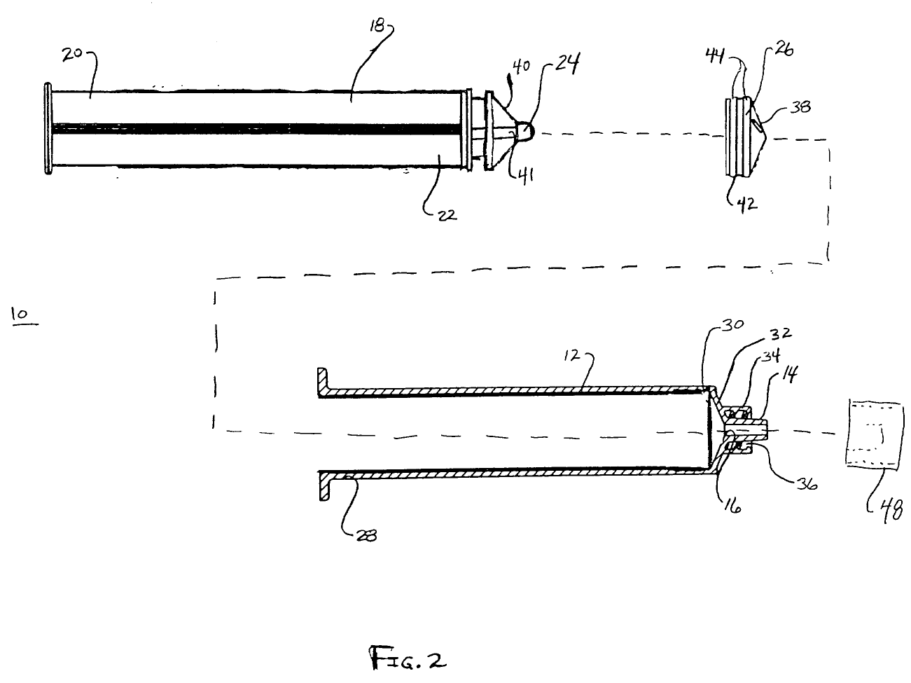

Syringe 10 includes a barrel 12 including a nozzle 14, which has an interior opening 16. Syringe 10 also includes a plunger rod 18 having a first end, such as, for example, proximal end 20 and a second end, such as, for example, distal end 22. Distal end 22 includes an extension 24 (shown in FIG. 2) dimensioned to ensure that a member, such as, for example, plunger tip member 26, disposed with distal end 22 and enclosing extension 24, abuts interior opening 16. Extension 24 advantageously prevents backflow or reflux of fluids such as blood, body fluids, flush solution, medication, etc. Extension 24 cooperates with nozzle 14, as will be discussed, to significantly reduce the rebound of plunger rod 18 during use.

Barrel 12 longitudinally extends from a proximal end 28 to a distal end 30. Distal end 30 includes a wall portion 32 (see FIG. 2) which tapers to support nozzle 14. Wall portion 32 has a conical configuration that engages a corresponding portion of distal end 22 of plunger rod 18. Distal end 22 may or may not include plunger tip member 26. It is contemplated that wall portion 32 may be variously configured for corresponding engagement with plunger rod 18, including for example, a planar surface, inverted, etc. It is further contemplated that wall portion 32 may have varying degrees of taper, such as, for example, acute, obtuse, or, alternatively may be substantially planar.

Nozzle 14 and a collar 34 of barrel 12 define a cavity 36 therebetween. The surface of collar 34 defining cavity 36 is threaded for attachment to a needle hub, luer connection, etc. It is contemplated that distal end 30 of barrel 12 may alternatively be variously configured for attachment such as, for example, slip (friction) fit, adhesive, etc. Nozzle 14 extends distally from distal end 30 for mounting with a needle hub, luer fitting, etc.

Proximal end 28 of barrel 12 is configured for slidable receipt and support of plunger rod 18. Plunger rod 18 is manipulated for slidable movement within barrel 12. Although shown as a manually manipulated syringe 10, it is contemplated that movement of plunger rod 18 relative to barrel 12 may be controlled through motorized mechanisms, electronic components, etc. It is envisioned that barrel 12 may be variously dimensioned and have alternative configurations such as, cylindrical, rectangular, etc.

Plunger tip member 26 encloses distal end 22 of plunger rod 18 and is fabricated from a flexible material such as, for example, rubber, etc. It is contemplated that plunger tip member 26 may have varying degrees of flexibility to provide rigidity, according to a particular syringe application. It is further contemplated that plunger rod 18 may not include a plunger tip member and that distal end 22 correspondingly engages wall portion 32. Plunger tip member 26 is configured to engage wall portion 32 and interior opening 16 of nozzle 14 to force fluid through nozzle 14 and a needle cannula, luer tip, etc. during a procedure.

Plunger tip member 26 has a tranverse wall 38 that encloses a distal face 40 of plunger rod 18. A cylindrical sidewall 42 extends along distal end 22 in a proximal direction. Transverse wall 38 is tapered in a conical configuration for corresponding engagement with wall portion 32 and has a uniform thickness. Transverse wall 38 is configured to correspondingly engage tapered distal face 40 and may alternatively be planar, non-uniform, inverted, etc., according to the requirements of a particular syringe application.

Distal face 40 includes struts 41 that engage and provide support for plunger tip member 26. It is envisioned that distal face 40 may be solid, inverted, parabolic, etc. Extension 24 and struts 41 force transverse wall 38 distally facilitating expulsion of fluid through interior opening 16 upon manipulation of plunger rod 18, as will be discussed. Sidewall 42, substantially encloses distal face 40 and a sufficient portion of distal end 22 to seal the inner chamber of barrel 12 during manipulation plunger rod 18. Wipers 44 disposed circumferentially about sidewall 42 facilitate the seal to thereby expel fluid through nozzle 14 and prevent leakage of fluid proximal of distal end 22.

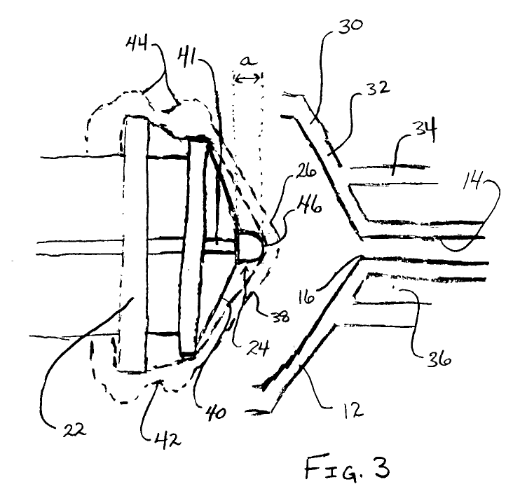

Distal face 40 includes extension 24 projecting distally therefrom. Extension 24 defines a distal portion 46, as shown in FIG. 3. Distal portion 46 is dimensioned for selective engagement of plunger tip member 26 with interior opening 16.

Extension 24 has a longitudinal length, such as, for example, shaft portion a, which extends to distal portion 46. Distal portion 46 is spherical and dimensioned to abut interior opening 16. Distal portion 46 is configured for receipt by interior opening 16 and has a diameter large enough to prevent passage into nozzle 14. Thus, during manipulation of plunger rod 18, shaft portion α extends a sufficient length in cooperation with the engagement of distal portion 46 with interior opening 16, to prevent compressive deformation of plunger tip member 26 between wall portion 32 and distal face 40. Shaft portion a recesses distal face 40, upon abutting engagement of extension 24 with interior opening 16, so that plunger tip member 26 does not elastically fill any void areas between distal face 40 and wall portion 32. This advantageously significantly reduces rebound of plunger rod 18 and the resulting drawbacks associated with reflux. It is contemplated that extension 24 may vary in length, such as, for example, in the range of 0.01″-0.100″ and distal portion 46 may have various geometric configuration, such as, for example, planar, diamond shaped, etc., according to the requirements of a particular syringe application and in accordance with the principles of the present disclosure. It is contemplated that extension 24 may be fabricated from polymerics, acrylics, Delrin®, etc.

In use, a syringe 10, similar to that described, is assembled, properly sterilized and otherwise prepared for storage, shipment and use. A syringe cap 48 is attached to distal end 30 of barrel 12 to prevent contamination and undesired engagement therewith. Syringe cap 48 may be threaded, interference fit, etc. with nozzle 14 and/or collar 34.

For example, syringe 10 is employed to flush an intravenous catheter (not shown). Nozzle 14 and collar 34 may be attached to a needle hub (not shown) for drawing and administering fluid with syringe 10, or alternatively, may be attached via a needless luer connection. Thus, syringe 10 is attached to a supply of flush solution and plunger rod 18 is manipulated proximally to draw the flush solution into barrel 12.

Nozzle 14 and collar 34 are attached to the intravenous catheter line for fluid communication therewith. Plunger rod 18 is manipulated distally to expel flush solution from barrel 12 and correspondingly flush the catheter line. Distal end 22 of plunger rod 18 approaches wall portion 32 of barrel 12 to expel the remaining flush solution from syringe 10. Distal portion 46 of extension 24 abuts interior opening 16, as discussed. Shaft portion a recesses distal end 22 such that distal face 40 does not undesirably deform the flexible material of plunger tip member 26 with wall portion 32. This prevents plunger tip member 26 from filling any void areas created between distal face 40 and wall portion 32 with elastic material that may cause rebound of plunger rod 18. Thus, the configuration at extension 24 advantageously prevents rebound of plunger tip member 26 and the associated drawbacks of reflux. It is envisioned that syringe 10 may also be employed for administration of medication, injections, etc.

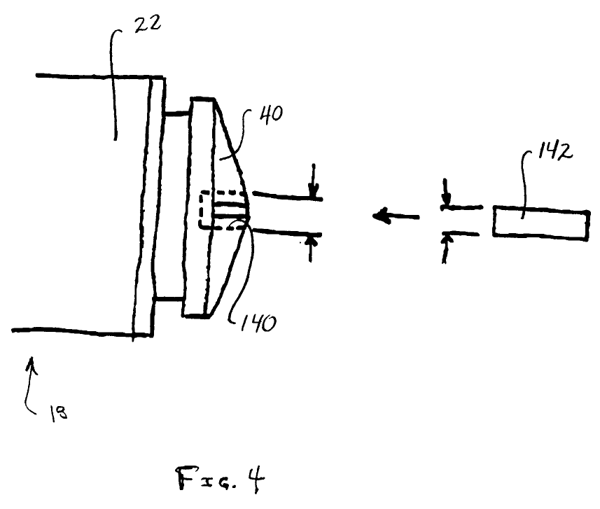

Referring to FIG. 4, an alternate embodiment of distal end 22 of plunger rod 18, similar to that described above, is shown. Distal face 40 includes a cavity 140 (shown in phantom) configured for receipt of an extension 142, similar to extension 24 described above. Extension 142 is separately fabricated and mounted with cavity 140. Extension 142 may be fixed with cavity 140 via adhesive, interference fit, etc., according to the requirements of a particular syringe application. It is envisioned that extension 142 and cavity 140 have substantially equal diameter, such as, for example, 0.10″, and are press fit for mounting. It is further envisioned that distal end 22 and extension 142 may be fabricated from similar or dissimilar materials.

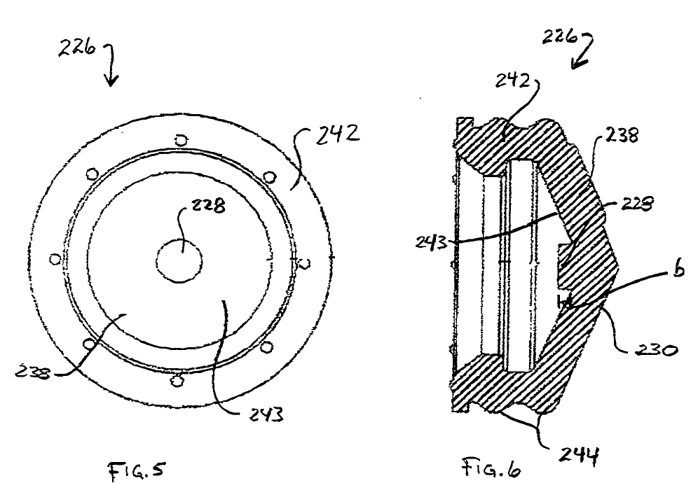

Referring to FIGS. 5 and 6, a plunger tip member 226 is shown, which is employed with an alternate embodiment of syringe 10, similar to that described with regard to FIGS. 1-4. In this embodiment, distal end 22 (FIG. 2) of plunger rod 18 does not include extension 24. Plunger tip member 226 includes an inner extension 228 dimensioned to ensure that plunger tip member 226, disposed with distal end 22, abuts interior opening 16 (FIG. 3) of nozzle 14. Inner extension 228 advantageously prevents backflow or reflux of fluids such as blood, body fluids, flush solution, medication, etc. Inner extension 228 cooperates with distal face 40 (FIG. 3) of plunger rod 18, as will be discussed, to significantly reduce the rebound of plunger rod 18 during use.

Plunger tip member 226 encloses distal end 22 of plunger rod 18 and is fabricated from a flexible material such as, for example, rubber, etc. It is contemplated that plunger tip member 226 may have varying degrees of flexibility to provide rigidity, according to a particular syringe application. It is further contemplated that plunger rod 18 may include a depression or extension disposed with distal face 40 that cooperates with inner extension 228. Plunger tip member 226 has an outer surface 230 that is configured to engage wall portion 32 (FIG. 3) and interior opening 16 of nozzle 14 to force fluid through nozzle 14 and a needle cannula, luer tip, etc. during a procedure.

Plunger tip member 226 has a tranverse wall 238 that encloses distal face 40. A cylindrical sidewall 242 extends along distal end 22 in a proximal direction. Transverse wall 238 is tapered in a conical configuration for corresponding engagement with wall portion 32 and has a substantially uniform thickness. Transverse wall 238 includes an inner surface 243 that is configured to correspondingly engage tapered distal face 40 and may alternatively be planar, non-uniform, inverted, etc., according to the requirements of a particular syringe application.

Sidewall 242 substantially encloses distal face 40 and a sufficient portion of distal end 22 to seal the inner chamber of barrel 12 during manipulation plunger rod 18. Wipers 244, disposed circumferentially about sidewall 242, facilitate the seal to thereby expel fluid through nozzle 14 and prevent leakage of fluid proximal of distal end 22.

Inner extension 228 has a longitudinal length, such as, for example, shaft portion b. Inner extension 228 is cylindrical and dimensioned to engage distal face 40. Inner extension 228 has a diameter large enough to prevent passage into nozzle 14 so that outer surface 230 of plunger tip member 226 abuts interior opening 16. Thus, during manipulation of plunger rod 18, shaft portion b extends a sufficient length to prevent compressive deformation of plunger tip member 226 between wall portion 32 and distal face 40, similar to extension 24 described above. Shaft portion b recesses distal face 40, upon engagement therewith and engagement of outer surface 230 with interior opening 16, so that plunger tip member 226 does not elastically fill any void areas between distal face 40 and wall portion 32. This advantageously significantly reduces rebound of plunger rod 18 and the resulting drawbacks associated with reflux. Inner extension 228 may be monolithically formed with plunger tip member 226 or integrally connected thereto. It is contemplated that inner extension 228 may be fabricated from polymerics, acrylics, Delrin®, etc.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A syringe comprising:
a barrel including a nozzle having an interior opening; and
a plunger rod having a first end and a second end, the second end including an extension dimensioned to ensure that a member disposed with the second end and enclosing the extension abuts the interior opening.

2. A syringe as recited in claim 1, wherein the interior opening has a cylindrical configuration.

3. A syringe as recited in claim 2, wherein the extension is dimensioned for receipt by the interior opening in an abutting relationship.

4. A syringe as recited in claim 1, wherein the barrel includes a distal wall that supports the nozzle and the plunger rod includes a correspondingly configured distal face.

5. A syringe as recited in claim 4, wherein the extension includes a longitudinal length and diameter such that upon engagement of the member with the interior opening, the length and diameter of the extension prevents compressive deformation of the member between the distal wall and the distal face.

6. A syringe as recited in claim 1, wherein the second end of the plunger rod has a tapered configuration for supporting the extension.

7. A syringe as recited in claim 1, wherein the member has a transverse wall and a cylindrical sidewall extending along the second end of the plunger rod.

8. A syringe as recited in claim 7, wherein the transverse wall is tapered.

9. A syringe as recited in claim 7, wherein the transverse wall has a substantially uniform thickness.

10. A syringe as recited in claim 1, wherein the extension is monolithically formed with the second end of the plunger rod.

11. A syringe as recited in claim 1, wherein the extension is integrally connected with the second end of the plunger rod.

12. A syringe as recited in claim 1, wherein the member is at least partially flexible.

13. A syringe comprising:
a barrel extending from a proximal end to a distal end thereof, the distal end including a wall portion tapered to support a nozzle, the nozzle defining an interior opening;
a plunger rod configured for slidable movement within the barrel and having a first end and a second end, the second end including an extension projecting distally therefrom, the extension defining a distal portion; and
a member defining a transverse wall and a side wall extending along the second end of the plunger rod, the transverse wall enclosing the extension;
wherein the distal portion of the extension is dimensioned for selective engagement of the member with the interior opening.

14. A syringe as recited in claim 13, wherein the extension includes a longitudinal length and diameter such that upon engagement of the member with the interior opening, the length and diameter of the extension prevents compressive deformation of the member between the wall portion of the barrel and a distal face of the plunger rod.

15. A syringe as recited in claim 13, wherein the second end of the plunger rod has a tapered distal face.

16. A syringe as recited in claim 13, wherein the transverse wall is substantially tapered.

17. A syringe as recited in claim 13, wherein the transverse wall has a substantially uniform thickness.

18. A syringe comprising:
a barrel extending from a proximal end to a distal end thereof, the distal end including a wall portion supporting a nozzle, the nozzle defining an interior opening;
a plunger rod configured for slidable movement within the barrel and having a first end and a second end, the second end including an extension means for preventing reflux of fluid back into the barrel; and
a member disposed with the second end and enclosing the extension means.

19. A syringe comprising:
a barrel including a nozzle having an interior opening; and
a member disposed with a distal end of a plunger rod being movable relative to the barrel, the member including an inner extension configured to engage the distal end and dimensioned to ensure that the member abuts the interior opening.

20. A syringe as recited in claim 19, wherein the inner extension includes a longitudinal length and diameter such that upon engagement of the member with the interior opening, the length and diameter of the extension prevents compressive deformation of the member between a distal wall of the barrel and a distal face of the plunger rod.
